(19) United States
(12) Patent Application Publication
(10) Pub. No.: US 2009/0227874 A1
(43) Pub. Date: Sep. 10, 2009

(54) HOLDER ASSEMBLY FOR A MEDICAL IMAGING INSTRUMENT

(75) Inventors: Jasjit S. Suri, Roseville, CA (US); Dinesh Kumar, Grass Valley, CA (US); Animesh Khemka, Grass Valley, CA (US)

Correspondence Address: MARSH, FISCHMANN & BREYFOGLE LLP, 8055 East Tufts Avenue, Suite 450, Denver, CO, 80237, US

(73) Assignee: EIGEN, INC., Grass Valley, CA (US)

(21) Appl. No.: 12/265,940

(22) Filed: Nov. 6, 2008

Related U.S. Application Data
(60) Provisional application No. 60/986,866, filed on Nov. 9, 2007.

(51) Int. Cl. A61B 8/14

(52) U.S. Cl. 600/459

(57) ABSTRACT

Provided herein are devices and methods for mounting variously configured medical imaging probes (“ultrasound probes”) to a positioning device for imaging applications. One aspect provides a probe holder that has a recessed surface adapted to securely support a particular ultrasound probe for interfacing the probe with a positioning device. Another aspect provides a probe holder that interconnects with a flexible joint to allow the probe holder to securely support a variety of different ultrasound probes and interface those probes with a positioning device.

### CROSS REFERENCE TO RELATED APPLICATIONS

This application claims the benefit of the filing date of U.S. Provisional Patent Application No. 60/986,866 entitled “Axis Alignment Method” and having a filing date of Nov. 9, 2007, the entire contents of which are incorporated herein by reference.

### FIELD

The present invention is directed to system, apparatus and method for holding and positioning a medical imaging instrument. More particularly, the invention relates to an apparatus adapted to hold a medical imaging instrument such that the instrument may be positioned, secured and/or rotated about at least one fixed axis by a positioning device.

### BACKGROUND

Doctors and other medical professionals often utilize medical imaging instruments to conduct non-invasive examinations. That is, medical imaging instruments, including X-ray, magnetic resonance (MR), computed tomography (CT), ultrasound, and various combinations of these instruments/techniques are utilized to provide images of internal patient structure for diagnostic purposes as well as for interventional procedures. These medical imaging instruments allow examination of internal tissue that is not readily examined during normal visual or tactile examination. Applications include imaging in the areas of urology, brachytherapy, cyrotherapy, photo-dynamic therapy, or a combination of these therapies and/or fusion-guided biopsies.

Medical imaging devices typically allow for generating three-dimensional (“3-D”) images of internal structures of interest. Such 3-D imaging may improve the accuracy and/or reliability of medical diagnosis. For instance, a medical imaging device may be utilized to generate a 3-D model or map of the prostate such that one or more biopsies may be taken from a desired location of the prostate. For purposes of prostrate imaging, a transrectal ultrasound-imaging device (e.g., a “TRUS probe” or “ultrasound probe”) may provide image acquisition and guidance. The ultrasound probe is a widely accepted technique for prostate applications due to its simplicity, high specificity, and real time nature. In such an application, the ultrasound probe or any other medical imaging device may be inserted into the rectum of a patient to generate an image. Such images may be utilized to take one or more biopsies from a prostate location of interest and/or implant radioactive seeds at one or more desired locations in a brachytherapy procedure.

In order to generate 3-D images, many medical imaging devices obtain a plurality of two-dimensional (“2-D”) images and combine these images together to form a 3-D image. Accordingly, movement of a medical imaging device between the acquisition of individual images makes it more difficult to properly align (e.g., register) the different images for purposes of generating accurate 3-D images. Thus, precise and repeatable placement and guidance of the medical imaging device is desirable to achieve accurate imaging and rendering of the applicable therapy.

Traditionally, a medical practitioner manipulates a medical imaging device by hand for medical image acquisition and/or treatment. That is, the medical practitioner manually guides the instrument. Such manual manipulation is suitable for many medical procedures. However, in instances where it is desirable to obtain multiple images for 3-D image generation, manual manipulation of the device may result in movement between images. Further, for biopsy and other treatment procedures it is desirable to know the relative location between an imaging instrument and a tissue area of interest. That is, it is important that the device directs an imaging field to a particular tissue location and remain stationary to allow for guiding a biopsy/treatment device to a tissue location within the imaging field. Relative movement between the imaging device and the tissue area of interest during imaging and/or biopsy/treatment may impede the successful performance of these procedures.

Accordingly, a number of probe holding and manipulating/positioning assemblies have been proposed. These assemblies generally involve a holder that interfaces with a medical imaging device such as an ultrasound probe. The holder is then interconnected to one or more mechanical armatures and/or actuators such that the probe may be mechanically positioned and/or rotated relative to an area of interest on a patient (a “positioning device”). While this approach is generally effective, a number original equipment manufactures (“OEMs”) produce handheld probes in the market (e.g., Asucon, Aloka, ATL, B&K, Diasonics, General Electric, Hewlett Packard, Hitachi, Interspec, Philips, Siemens, Toshiba, etc.). While these probes do have some commonalities, they do not have a standardized design. Each probe generally has an acquisition or insertion portion and a handle portion. Typically, the acquisition portion is offset at an angle from the handle portion for ergonomic purposes. That said, probes produced by different manufactures feature acquisition portions and handle portions of differing lengths, widths, configurations, and offsets. Because limiting the rotation of the probe about an axis defined by the acquisition portion of the probe (an “acquisition axis”) removes a degree of freedom from subsequent calculations used to register a sequence of 2-D images together to form a 3-D image, the lack of conformity between probes has resulted in the need for specialized positioning devices for differently configured ultrasound probes. Accordingly, prior positioning devices have required that a medical facility utilize a particular probe with a particular specialized positioning device.

### SUMMARY

The present invention provides systems and methods for interfacing variously configured medical imaging devices for imaging applications (e.g., TRUS probes or other ultrasound probes, biopsy needles, therapeutic devices, medical imaging devices, etc.) in a fixed positional relationship with a positioning device. Such positioning devices may be utilized to position medical imaging devices relative to a patient tissue of interest. Aspects of the present invention provide an apparatus for interfacing an ultrasound probe with such a positioning device.

The apparatus embodied by a first aspect includes a connector and a probe holder. The connector is adapted to be rotatively coupled to a positioning device such that the connector is operative to rotate relative to the positioning device about a rotational axis. The probe holder includes a recessed surface that is sized to receive and secure at least a portion of an ultrasound probe. In addition, the probe holder is coupled to the connector at a location that is spaced from the rotational axis, and as a result, the recessed surface is offset from the rotational axis. This offset may allow an acquisition portion of a probe placed in the recessed surface to be aligned with the rotational axis of the positioning device.

The connector may be coupled to both the positioning device and the probe holder in any appropriate manner that allows the connector, and thus the probe holder, to rotate about the rotational axis. For instance, the connector may be fixedly coupled to a mating shaft that is rotatively coupled to the positioning device so as to allow the positioning device to rotate the connector via the mating shaft. In addition, because the probe holder is coupled to the connector at a position that is spaced from the rotational axis, the probe holder may orbit the rotational axis when the positioning device is in operation.

In one arrangement, the apparatus may include an ultrasound probe disposed within the recessed surface of the probe holder. The ultrasound probe may have a handle portion that generally defines a handle axis and an acquisition portion that generally defines an acquisition axis. In some cases, the handle axis and the acquisition axis may be nonconcentric. However, when the handle portion is disposed and/or secured within the recessed surface, the rotational axis of the positioning device and the acquisition axis of the acquisition portion of the probe are substantially concentric. This concentric alignment allows the acquisition portion of the probe to rotate about the rotational axis substantially free of precession or wobble, which may provide for improved image registration.

In one arrangement, the recessed surface of the probe holder may be correspondingly shaped to the handle portion of the probe. When the recessed surface of a particular probe holder is correspondingly shaped to concentrically align a particular probe, different probes having different configurations may utilize different probe holders to interface with the positioning device. That is, each different probe may utilize a customized probe holder.

In another arrangement, the apparatus may include a flexible joint disposed between the connector and the probe holder. The flexible joint may permit the selective adjustment and/or alignment of the probe holder, and thus a secured probe, in relation to the rotational axis of the positioning device. That is, the flexible joint may allow for aligning an acquisition axis of the probe with the rotational axis of the positioning device. As a result, the flexible joint may allow for more general use of the probe holder, or allow for a single probe holder to be used with various probe configurations.

The flexible joint may include one or more rotational, revolving, gyratory, or other joints of any appropriate size, type, and/or configuration. For instance, the flexible joint may include one or more ball-and-socket joints, a ball-bar-socket joint, a binge joint, or any other appropriate joint that allows for adjustment within multiple degrees of freedom.

The apparatus may also include a locking mechanism adapted to lock the flexible joint in a desired position. For example, in one arrangement, one or more securement plates may be tightened about each ball-and-socket or other rotational joint so as to prevent further movement of the rotational joint. The securement plates may be tightened with threaded fasteners such as, for example, set screws, thumbscrews, cap screws, etc.

In another aspect of the present invention, an alignment fixture may be used to adjust a flexible joint of a probe holding apparatus to concentrically align a rotational axis of a positioning device with an acquisition axis of an ultrasound probe. In this aspect, the alignment fixture may include a base that supports a probe holding apparatus in a desired relationship to the rotational axis. In one arrangement, a connector of the apparatus may be fixedly positioned relative to the rotational axis, and a probe holder of the apparatus may then be adjusted. In such an arrangement, a probe holder may be coupled to the connecter via a flexible joint. The alignment fixture may also incorporate an indicator that provides an indication of the alignment of the acquisition axis of a probe disposed in the probe holder to the relational axis. For example, when an ultrasound probe is secured within the probe holder, a lever arm of such an indicator may engage the acquisition portion of the probe such that when the mating shaft rotates the probe, the indicator detects any precession in the acquisition portion of the probe. If precession is detected, the flexible joint may be adjusted until the acquisition portion of the probe rotates about the rotational axis substantially free of precession before the flexible joint is secured

### BRIEF DESCRIPTION OF THE DRAWINGS

FIG. 1A shows a cross-sectional view of a trans-rectal ultrasound imaging system as applied to perform prostate imaging.

FIG. 1B illustrates use of a positioning device to position an ultrasound imaging device to perform prostate imaging.

FIG. 2A illustrates two-dimensional images generated by the trans-rectal ultrasound imaging system of FIG. 1.

FIG. 2B illustrates a three-dimensional volume image generated from the two dimensional images of FIG. 2A.

FIG. 3 illustrates a side plan view of one embodiment of an ultrasound probe.

FIG. 4 illustrates a perspective view of one embodiment of a holder assembly for securing an ultrasound probe.

FIG. 5 illustrates a perspective view of an ultrasound probe secured within the holder assembly of FIG. 4.

FIG. 6 illustrates another embodiment of a holder assembly for securing an ultrasound probe.

FIG. 7 illustrates a perspective view of an ultrasound probe secured within the holder assembly of FIG. 6.

FIG. 8 illustrates a perspective view of a portion of a flexible joint.

FIG. 9 illustrates another perspective view of a portion of a flexible joint.

FIG. 10 illustrates a perspective view of the holder assembly and secured ultrasound probe of FIG. 7 as mounted within an alignment fixture.

FIG. 11 illustrates a detailed view of the biasing collar assemblies of the alignment fixture of FIG. 10.

### DETAILED DESCRIPTION

Reference will now be made to the accompanying drawings, which assist in illustrating the various pertinent features of the present disclosure. Although the present disclosure is described primarily in conjunction with transrectal ultrasound imaging for prostate imaging, it should be expressly understood that aspects of the present invention may be applicable to other medical imaging applications. In this regard, the following description is presented for purposes of illustration and description.

Systems and methods are disclosed that facilitate obtaining medical images and/or performing medical procedures. One embodiment provides a medical imaging device holder (i.e., probe holder or holder) that has a recessed surface adapted to securely support a particular ultrasound probe for interfacing the ultrasound probe in a fixed positional relationship with a positioning device. Another embodiment provides a probe holder that allows for more general use of a single holder assembly that is capable of securely supporting a variety of different ultrasound probes in a desired positional relationship with a positioning device. The probe holders of the present invention may be interfaced with a positioning device such that the acquisition axis of the supported probe may be rotated about the rotational axis of the positioning device in a manner substantially free of precession or wobble. In this regard, the supported probe may obtain multiple images in different angular positions for 3-D image generation. Because the positioning device securely supports the probe, there may be little or no probe movement between successive images, other than rotation about the rotational axis. Accordingly, successive images may more easily be registered together.

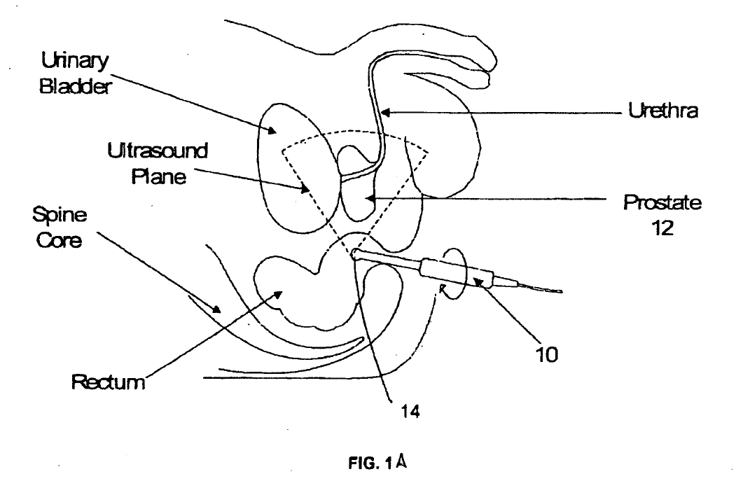

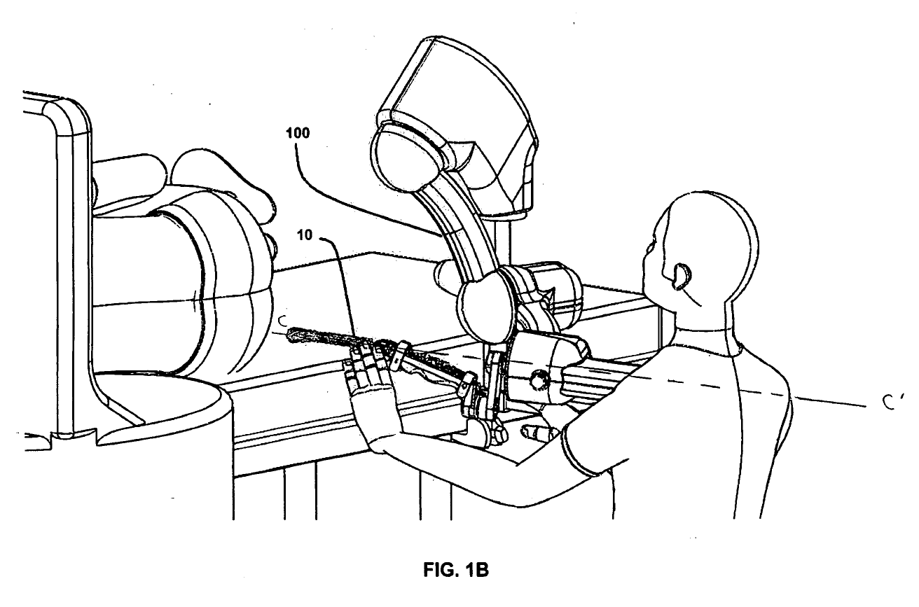

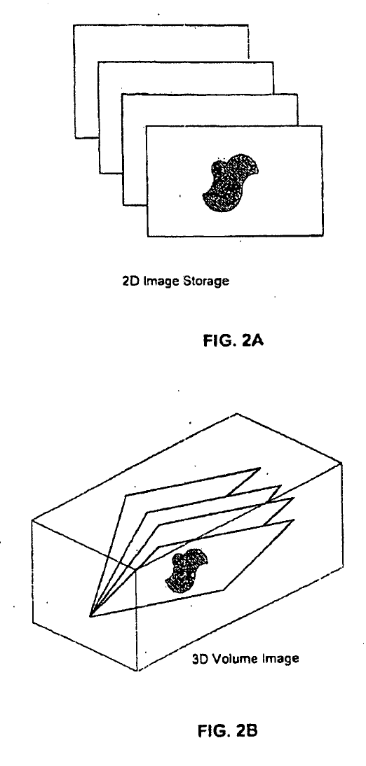

FIG. 1A illustrates a transrectal ultrasound probe being utilized to obtain a plurality of 2-D ultrasound images of the prostate 12. As shown, the probe 10 may be operative to automatically scan an area of interest. In such an arrangement, a user may rotate the acquisition portion 14 of the ultrasound probe 10 over an area of interest. Accordingly, the probe 10 may acquire plurality of individual images while being rotated over the area of interest. See FIGS. 2A-B. Each of these individual images may be represented as a 2-D image. See FIG. 2A. Initially, such images may be in a polar coordinate system. In such an instance, it may be beneficial for processing to translate these images into a rectangular coordinate system. In any case, the 2-D images may be combined to generate a 3-D image. See FIG. 1B.

As shown in FIG. 1A, the ultrasound probe 10 is a side-fire probe that generates ultrasound waves out of the side surface. However, it will be appreciated that end-fire scan probe may be utilized as well. In any arrangement, the probe 10 may also include a biopsy gun (not shown) that may be attached to the probe. The biopsy gun may include a spring driven needle that is operative to obtain a core from desired area within the prostate. In this regard, it may be desirable to generate an image of the prostate 12 while the probe 10 remains positioned relative to the prostate. If there is little or no movement between acquisition of the images and generation of the 3D image, the biopsy gun may be positioned to obtain a biopsy (or perform other procedures) of an area of interest within the prostate 12. However, manual manipulation of the probe 10 often results in relative movement between the probe and the prostate 12 between subsequent images and/or as a biopsy device is guided toward an area of interest.

Accordingly, it is desirable to minimize relative movement between the probe 10 and the prostrate 12 (i.e., precession, wobble or any other rotational movement of the probe about a fixed axis for image acquisition). It is also often desirable for probe 10 to remain fixed relative to the prostrate 12 during biopsy or other treatment procedures such that the desired tissue locations may be targeted accurately. To achieve such fixed positioning of probe 10, it is often desirable to interface the probe 10 with a positioning device such as the exemplary positioning device 100 shown in FIG. 1B. The positioning device 100 maintains the probe 10 in a fixed position relative to the prostate 12 and provides location information (e.g., frame of reference information) for use with an acquired image. In this regard, location outputs from the positioning device 100 may be supplied to a computer and/or imaging device. Likewise, the output of the probe 10 may be provided to the computer and/or imaging device, and the computer and/or imaging device may utilize this information to produce an output (e.g., display) of the imaged object (e.g., prostate).

The present invention may be used to interface an ultrasound probe with various positioning devices of the type discussed above. One exemplary positioning device is set forth in International Application Number PCT/CA2007/001076, entitled Apparatus for Guiding a Medical Tool. Another is set forth in U.S. application Ser. No. 11/850,482, entitled Tracker Holder Assembly, the contents of which are fully incorporated herein by reference.

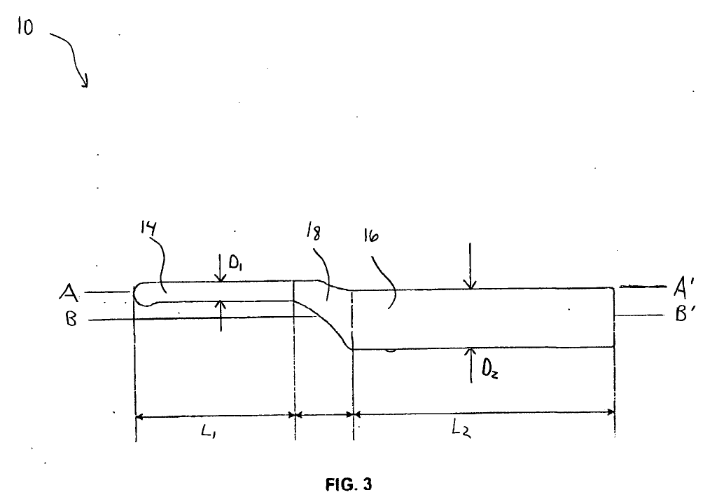

In order to utilize the probe 10 with the positioning device 100 as illustrated in FIG. 1B, it is necessary to secure the probe 10 to the positioning device. That is, there must be an interface between the probe 10 and the positioning device 100. The fact that probes made by different probe manufacturers have different dimensions complicates the task of interfacing the ultrasound probe 10 with the positioning device 100. For instance, FIG. 3 illustrates an exemplary ultrasound probe 10. In this embodiment, the probe 10 includes an acquisition portion 14 having a first length L₁ and a first diameter D₁ (i.e., insertion diameter). The acquisition portion defines an acquisition axis AA′. The probe 10 also includes a handle portion 16 having a second length L₂ and a second diameter D₂. The handle portion defines a handle axis BB′. Generally, the acquisition axis AA′ and the handle axis BB′ are offset such that the axes are nonconcentric. The probe 10 may also have a transition portion 18 between the insertion portion 14 and the handle portion 16. The combined lengths of components 14, 16 and 18 define the overall length of the probe 10.

As discussed above, the dimensions (e.g., lengths and/or diameters) of any or all of these components 14, 16 and 18 may vary between probes of different manufactures. Further, these components may be tapered and/or set at an angle to one another. Therefore, to interface different probes 10 to a common positioning device 100 typically requires individual probe interfaces.

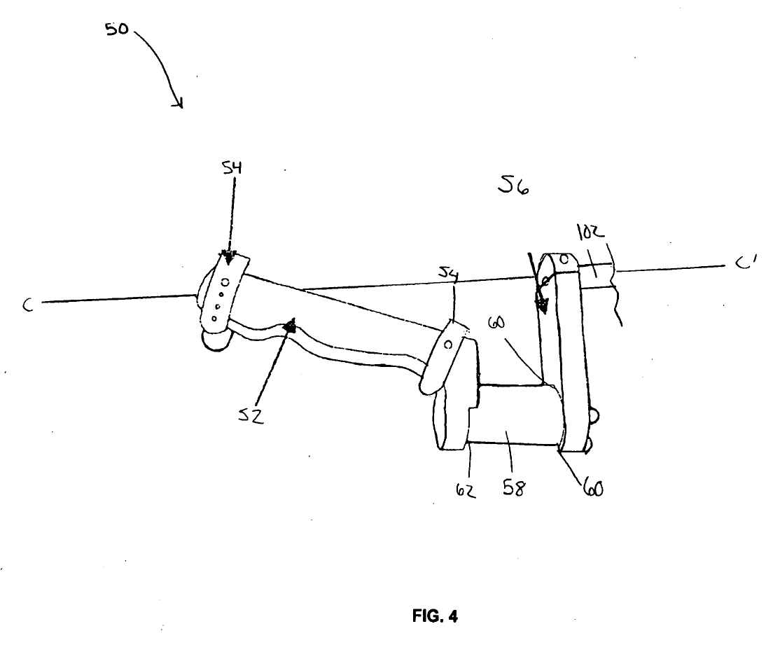

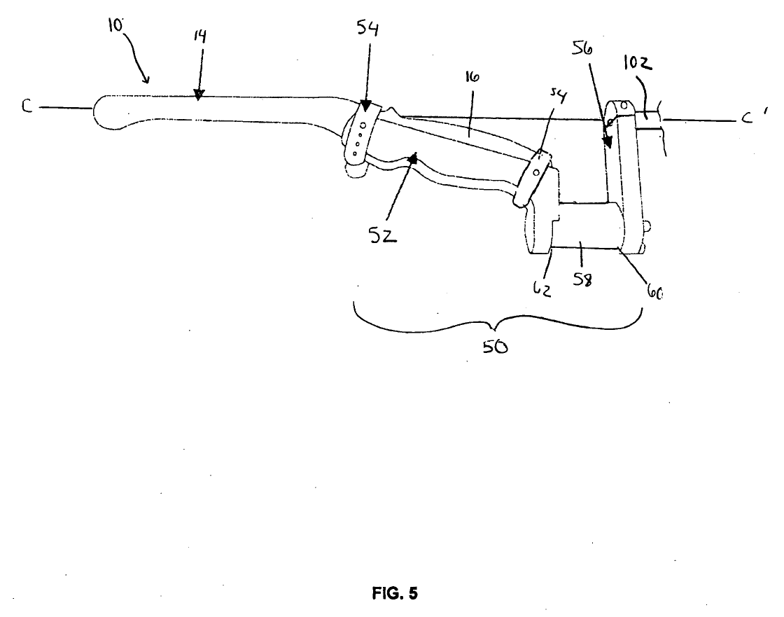

Accordingly, FIGS. 4-5 illustrate one embodiment of a probe holder assembly 50 that may be used to interface differently configured ultrasound probes with a positioning device 100 (FIG. 1B). FIG. 4 illustrates the holder assembly 50 alone, while FIG. 5 shows the ultrasound probe 10 secured within the holder assembly 50. While the holder assembly 50 is described in conjunction with the positioning device 100 (FIG. 1B), it should be appreciated that the holder assembly 50 may be used with any appropriate positioning device.

In this embodiment, the holder assembly 50 includes a probe holder 52 and one or more operably connected straps 54 for cinching or securing the handle portion 16 of the ultrasound probe 10 within the probe holder 52, as shown in FIG. 5. The straps 154 may be attached in any appropriate manner, including via threaded or self-tapping fasteners or a snap or press fit. The probe holder 52 forms a recessed surface that, in the present embodiment, is correspondingly shaped to the handle portion 16 of the ultrasound probe 10 such that when the probe 10 is disposed within the probe holder 52 and the holder assembly 50 interfaces with the positioning device 100 (FIG. 1B), the acquisition axis AA′ (FIG. 3) defined by the acquisition portion 14 of the probe 10 is concentrically aligned with a fixed rotational axis CC′ of the positioning device 100 (FIG. 1B). This allows the acquisition portion 14 of the probe to rotate about a fixed axis CC′, which may provide for improved image registration.

In the embodiment of FIGS. 4 and 5, differently configured ultrasound probes from different OEMs will utilize different probe holders, with each forming a correspondingly configured recessed surface. That is, the recessed surfaces may be individually tailored for individual probes. While different probes 10 utilize different probe holders 52, each probe holder 52 utilizes a standardized interface that allows the probe holder 52 and a supported probe 10 to engage with the remainder of the holder assembly 50. In one embodiment, the selected probe holder 52 is fixably interconnected to a connector 56 through a rod 58. That is, in this embodiment, the probe holder 52, the rod 58, and the connector 56 may be formed of a single, continuous piece. In another embodiment, the selected probe holder 52 is releaseably interconnected to a connector 56 through a rod 58 having a proximal end 60 that is received within a mating aperture (not shown) within the connector 56 and a distal end 62 that is received within a mating aperture (not shown) of the probe holder 52. It will be appreciated that these apertures may be threaded, utilize a snap-fit configuration or a press fit configuration, or utilize threaded or self-tapping fasteners (e.g., setscrews) to secure the rod 58 therein. It will also be appreciated that any connecting member (e.g., a bar, a plate, etc.) may replace the rod 58.

A mating shaft 102 of the positioning device 100 (FIG. 1B) defines the rotational axis CC′ of the positioning device 100. The connector 56 is adapted to couple with the mating shaft 102 in any appropriate manner that allows the connector 56 to rotate with the mating shaft 102 about the rotational axis CC′ during operation of the positioning device 100. Accordingly, when the connector 56 is interconnected to the mating shaft 102, the holder assembly 50, and thus the supported probe 10, may be rotated about the rotational axis CC′ of the mating shaft 102. Notably, when the probe 10 is secured within a selected customized probe holder 52 of the holder assembly 50, the rotational axis CC′ of the mating shaft 102 is concentrically aligned with the acquisition axis AA′ (FIG. 3) of the probe 10 such that during operation, the positioning device 100 rotates the acquisition portion 14 of the supported probe 10 about the rotational axis CC′ in a manner that is substantially free of precession or wobble.

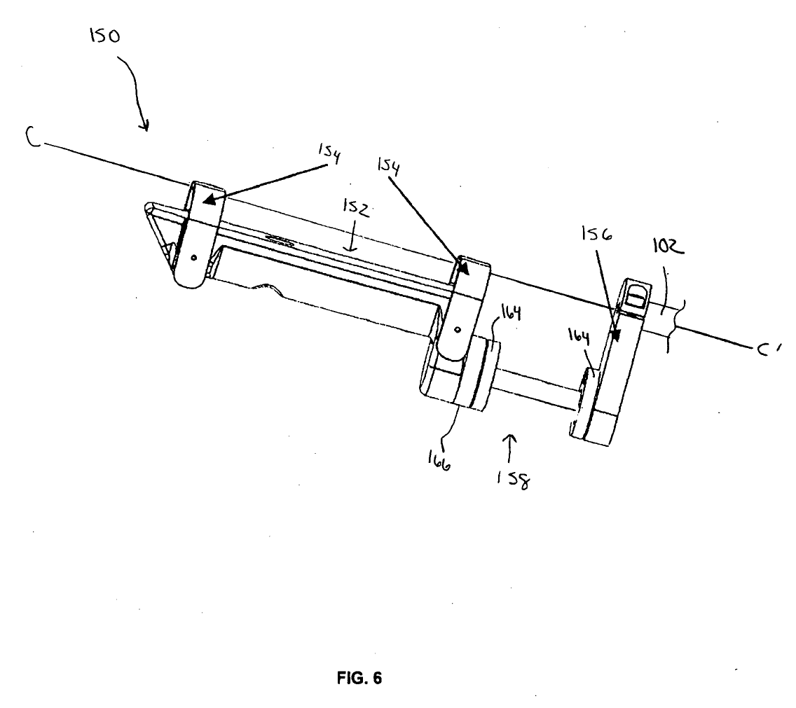

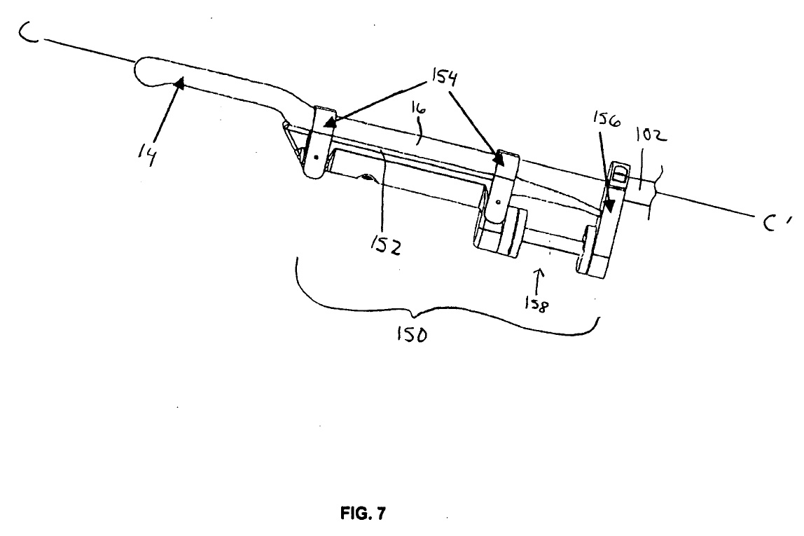

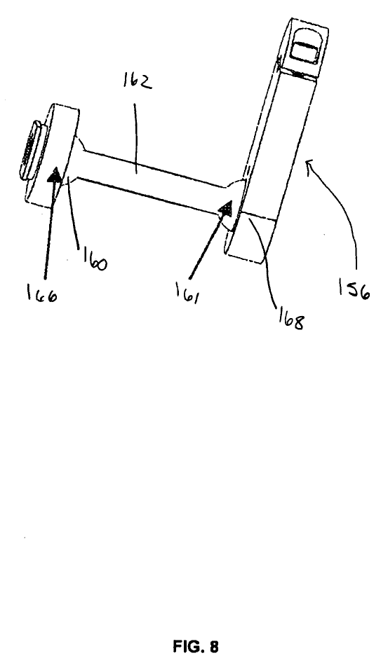

FIGS. 6-7 illustrate another embodiment of a holder assembly 150 for attachment to and use with the positioning device 100. Like holder assembly 50 discussed above, a connector 156 of the holder assembly 150 may be rotatively coupled with the mating shaft 102 of the positioning device 100 in any appropriate manner that allows the holder assembly 150, and thus the secured probe 10, to rotate with the mating shaft 100 substantially without precession. While the probe holder assembly 150 is described in conjunction with the positioning device 100 (FIG. 1B), it should be appreciated that the holder assembly 150 may be used with any appropriate positioning device.

In this embodiment, the holder assembly 150 incorporates a flexible joint 158, which allows for more general use of the probe holder 152. Specifically, the holder assembly 150 includes a probe holder 152 having a recessed surface that can accommodate a variety of different ultrasound probes 10. One or more straps 154 or clamps are operably connected with the probe holder 152 such that the selected probe 10 may be cinched or otherwise secured within the probe holder 152, as shown in FIG. 7. The straps 154 may be attached in any appropriate manner, including via threaded or self-tapping fasteners or a snap or press fit.

Because the probe holder 152 does not have a custom recessed surface for each particular ultrasound probe 10 (i.e., the recessed surface is not configured for each particular ultrasound probe), the holder assembly 150 includes the flexible joint 158 between the probe holder 152 and the connector 156. The flexible joint 158 allows the recessed surface (which engages the probe handle) of the probe holder 152 to be adjusted to bring the acquisition axis AA′ (FIG. 3) of the secured probe 10 into concentric alignment with the rotational axis CC′ of the positioning device 100 (FIG. 1B). As will be discussed below, the flexible joint also includes a locking mechanism to secure the probe holder 152, and thus the secured probe 10, in a concentrically aligned position.

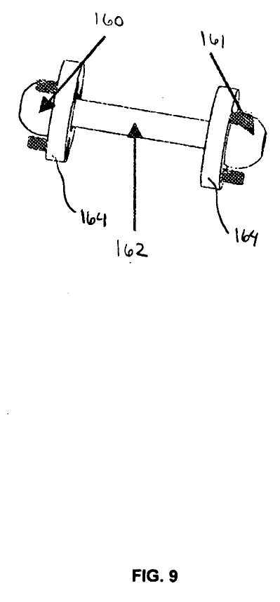

FIGS. 8-9 illustrate one embodiment of the flexible joint 158 in greater detail. In this embodiment, the flexible joint 158 includes a ball-bar-socket joint (e.g., two ball-and-socket joints joined by a bar) that connects the probe holder 152 with the connector 156. Specifically, a first ball 160 mates with a corresponding socket (not shown) formed within a proximal end 166 of the probe holder 152. It should be appreciated that the socket may be formed within a continuous portion of the probe holder 152 or a separate portion (e.g., a plate, a flange, a block, etc.) that is fixably attached to the probe holder 152 via any appropriate method of attachment (e.g., threaded fasteners, adhesive, welding, press fit, snap fit, etc.).

A second ball 161 mates with a socket (not shown) formed within an offset portion 168 of the connector 156. When disposed within the sockets, the first and second balls 160, 161 may be adjusted through nearly unlimited degrees of freedom in order to concentrically align the acquisition axis AA′ (FIG. 3) of the supported probe 10 (FIG. 7) with the rotational axis CC′ of the positioning device 100 (FIG. 1B).

To secure the balls 160, 161 in their respective aligned positions within the sockets, two securement plates 164 (FIG. 9) may be disposed about the bar 162 and tightened about each mated ball and socket using threaded fasteners that extend through the securement plates 164 into the material surrounding the sockets on the proximal portion 166 of the probe holder 152 and the offset portion 168 of the connector 156 (FIG. 8), respectively. The balls, sockets, and securement plates may be of any appropriate size, type, and/or configuration such that when tightened, the securement plates 164 compress the balls 160, 161 against the walls of the sockets, thereby preventing further rotation of the balls 160, 161 within the sockets and locking the probe holder 152 and supported probe 10 in an aligned position.

While the flexible joint 158 has been described as having two ball-and-socket joints, it should be understood that the flexible joint 158 may include additional or fewer rotative or other joints of any appropriate size, type, and/or configuration.

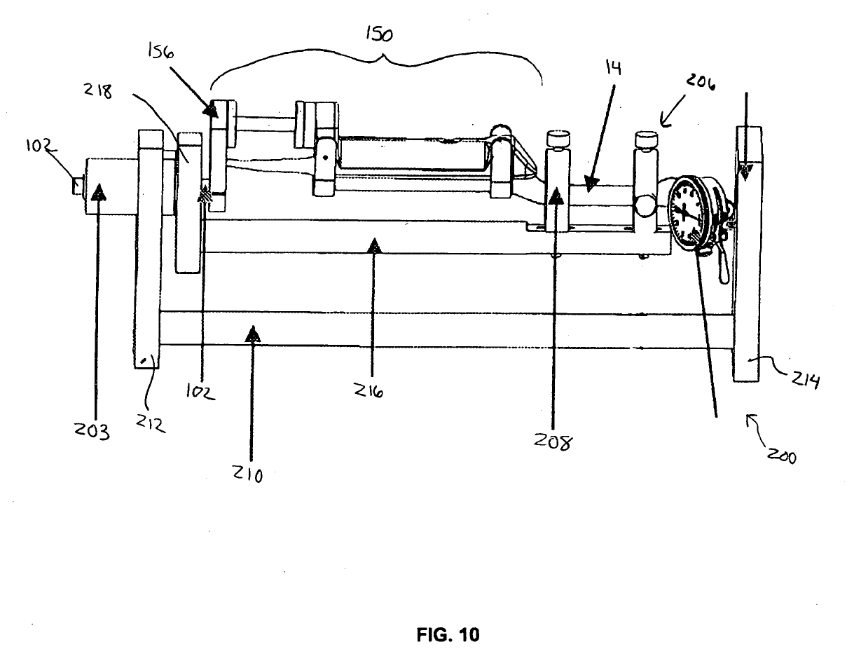

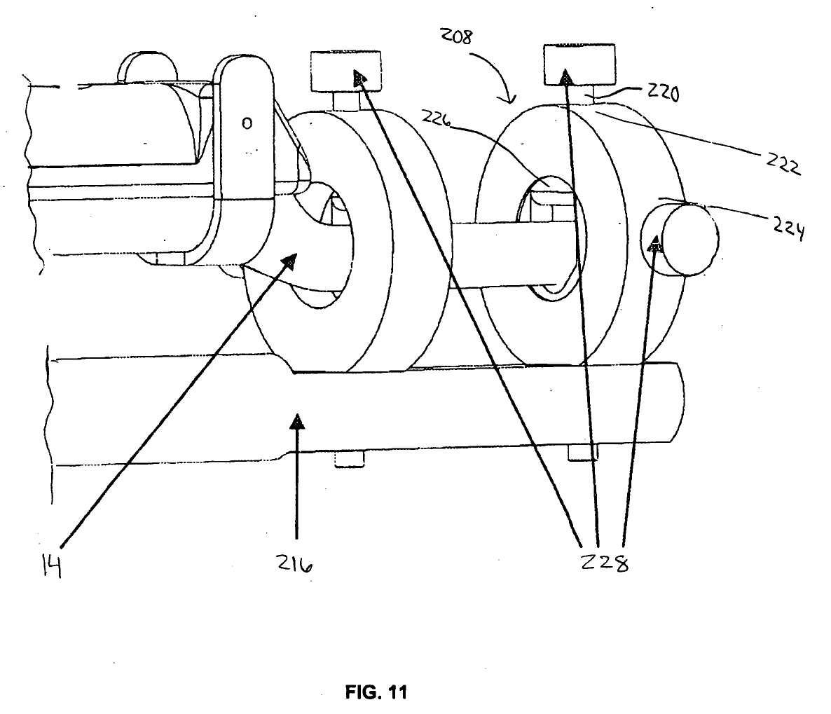

In some instances, an alignment fixture may be used to adjust the flexible joint 158 as discussed above. FIG. 10 illustrates the ultrasound probe 10 as secured within the holder assembly 150 and mounted upon one embodiment of an alignment fixture 200. To allow for adjustment, the flexible joint 158 of the holder assembly 150 remains loosely connected (i.e., the securement plates 164, 165 are not fully tightened about the ball-and-socket joints) until the probe 10 has been aligned, as discussed below.

The alignment fixture 200 includes a base having a first bar 210 that fixably connects a first end 112 and a second end 214. The first end 212 includes a mating shaft holder 203 that fixably couples to a second bar 216 through a disk 218. The mating shaft holder 203 also receives the mating shaft 102 of the positioning device 100 (FIG. 1B), which, in turn, is coupled with the connector 156 of the holder assembly 150 in any appropriate manner so as to allow the mating shaft 102 to rotate the connector 156 of the holder assembly 150, and thus the secured probe 10.

The acquisition portion of the secured probe 10 extends through two apertures 216 formed by corresponding biasing collar assemblies 206 that are coupled to a distal end of the second bar 216. The biasing collar assemblies 206 allow for the selective and incremental positioning of the acquisition portion 14 of the supported probe 10. As shown in further detail in FIG. 11, each collar assembly 206 includes a collar 208 that is adapted to receive the acquisition portion 14 of the supported probe 10. An adjustment screw 220 threadably engages with both a top portion 222 and a side portion 224 of the collar 208. The adjustment screws 220 may be of sufficient length to extend into an aperture 226 formed by the collar 208 such that the adjustment screws 220 contact the acquisition portion 14 of the secured probe 10. The adjustment screws 220 may be threaded fasteners of any appropriate size, type, and/or configuration that allows the screws 220 to extend through the collar 208 and into the aperture 226. Thumb knobs 228 may be attached to heads of the adjustment screws 220 to allow an operator to adjust the screws by hand.

In addition, biasing members (not shown) are disposed within the aperture 226 and opposite the adjustment screws 222. The biasing members deflect slightly to accommodate the acquisition portion 14 of the supported probe 210. This slight deflection causes the biasing members apply a compressive force between the acquisition portion 14 and the adjustment screws 220. As a result, the biasing members deflect further when the acquisition portion 14 shifts in response to incremental movement of the adjustment screws 218, thereby allowing the axis AA′ of the acquisition portion 14 of the probe 10 to be concentrically aligned with the rotational axis CC′ of the mating shaft. It should be appreciated that the biasing members may be coil springs, leaf springs, or any other bias springs of appropriate size, type, and/or configuration.

To gauge probe alignment, the alignment fixture 200 may also include a dial indicator 230 having a lever arm (not shown) that engages the acquisition portion 14 of the supported probe 10. As the mating shaft 102 rotates the supported probe 10, the indicator 230 detects any precession or wobble in the acquisition portion 14 of the probe 10. If precession is detected, the biasing collar assemblies 206 may be adjusted to bring the acquisition portion 14 of the probe 10 into concentric alignment with the shaft 102, as discussed above. Once aligned, the securement plates 164, 165 of the flexible joint may be tightened about the ball-and-socket joints to secure the probe 10 within the holder assembly 150 in an aligned position.

It will be appreciated that though a finite number of exemplary embodiments have been discussed herein, the various elements of those embodiments may be used separately or combined together. For example, the customized probe holder 52 may be used in conjunction with the holder assembly 150 having the flexible joint 158.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described above are further intended to explain best modes known of practicing the invention and to enable others skilled in the art to utilize the invention in similar or other embodiments and with various modifications required by the particular application(s) or use(s) of the present invention. It is intended that the appended claims be construed to include alternative embodiments to the extent permitted by the prior art.

What is claimed:

1. An apparatus for interfacing an ultrasound probe with a positioning device, comprising:
a connector adapted to be rotatively coupled to a positioning device, wherein said connector is operative to rotate relative to the positioning device about a rotational axis; and
a probe holder having a recessed surface sized to receive and secure at least a portion of an ultrasound probe, wherein said probe holder is coupled to said connector at a location spaced from said rotational axis, and wherein said recessed surface is offset from said rotational axis.

2. The apparatus of claim 1, further comprising an ultrasound probe disposed in said recessed surface having a handle portion that generally defines a handle axis and an acquisition portion that generally defines an acquisition axis, wherein said handle axis and said acquisition axis are nonconcentric.

3. The apparatus of claim 2, wherein said recessed surface of said probe holder is correspondingly shaped to said handle portion of said ultrasound probe.

4. The apparatus of claim 2, wherein said recess surface defines a receiving bore for said handle portion, and wherein a longitudinal axis of said recessed surface is nonconcentric with said rotational axis.

5. The apparatus of claim 2, wherein when said handle portion of said ultrasound probe is disposed within said recessed surface, said rotational axis and said acquisition axis are substantially concentric, and wherein said acquisition portion of said ultrasound probe rotates about said rotational axis substantially free of precession.

6. The apparatus of claim 1, further comprising at least one strap to secure said at least a portion of said ultrasound probe within said recessed surface.

7. The apparatus of claim 1, wherein said connector and the positioning device are rotatively coupled via a shaft having a first end that is coupled to the positioning device and a second end that is coupled to said connector, and wherein said shaft and said rotational axis are concentric.

8. The apparatus of claim 1, further comprising a flexible joint disposed between said connector and said probe holder, wherein said flexible joint permits selective positioning of said recessed surface relative to said rotational axis.

9. The apparatus of claim 8, wherein said flexible joint comprises a locking mechanism to fix said recessed surface in a position relative to said rotational axis.

10. The apparatus of claim 8, wherein said flexible joint comprises at least one ball-and-socket joint, wherein said at least one ball-and-socket joint is selectively securable by one or more adjustment screws.

11. The apparatus of claim 8, wherein said flexible joint comprises a first ball-and-socket joint and a second ball-and-socket joint, wherein said first and second ball-and-socket joints are interconnected via an adjustment bar.

12. An apparatus for interfacing an ultrasound probe with a positioning device, comprising:
a connector adapted to be rotatively coupled to a positioning device, wherein said connector is operative to rotate relative to the positioning device about a rotational axis;
a probe holder having a recessed surface, wherein said probe holder is coupled to said connector at a location spaced from said rotational axis, and wherein said recessed surface is offset from said rotational axis; and
an ultrasound probe having a handle portion that generally defines a handle axis and an acquisition portion that generally defines an acquisition axis, wherein said handle axis and said acquisition axis are nonconcentric, wherein at least a portion of said handle portion is disposed within said recessed surface and said acquisition axis is concentrically aligned with said rotational axis.

13. The apparatus of claim 12, wherein said recessed surface of said probe holder is correspondingly shaped to said handle portion of said ultrasound probe.

14. The apparatus of claim 12, further comprising at least one strap to secure said handle portion of said ultrasound probe within said recessed surface.

15. The apparatus of claim 12, wherein said connector and the positioning device are rotatively coupled via a shaft having a first end that is coupled to the positioning device and a second end that is fixably coupled to said connector, and wherein said shaft and said rotational axis are concentric.

16. An apparatus for interfacing an ultrasound probe with a positioning device, comprising:
a connector adapted to be rotatively coupled to a positioning device, wherein said connector is operative to rotate relative to the positioning device about a rotational axis;
a probe holder having a recessed surface sized to receive and secure at least a portion of an ultrasound probe, wherein said probe holder is coupled to said connector at a location spaced from said rotational axis, and wherein said recessed surface is offset from said rotational axis; and
a flexible joint disposed between said connector and said probe holder, wherein said flexible joint permits selective positioning of said recessed surface relative to said rotational axis.

17. The apparatus of claim 16, wherein said flexible joint comprises a locking mechanism to fix said probe holder and said recessed surface in a position relative to said rotational axis.

18. The apparatus of claim 16, wherein said flexible joint comprises at least one ball-and-socket joint, wherein said at least one ball-and-socket joint is secured by one or more adjustment screws.

19. The apparatus of claim 16, wherein said connector and the positioning device are rotatively coupled via a shaft having a first end that is coupled to the positioning device and a second end that is coupled to said connector, and wherein said shaft and said rotational axis are concentric.
